(19) United States
(12) Patent Application Publication
(10) Pub. No.: US 2026/0311799 A1
(43) Pub. Date: Oct. 8, 2026

(54) CLEARANCE PREDICTION ACCORDING TO ANTIBODY PROPERTY ANALYSIS

(71) Applicant: Genentech, Inc., South San Francisco, CA (US)

(72) Inventors: Parisa MAZROOEI, Foster City, CA (US); Daniel Saldinger O'NEIL, Oakland, CA (US); Saroja RAMANUJAN, San Mateo, CA (US)

(21) Appl. No.: 19/481,390

(22) PCT Filed: Apr. 29, 2024
(86) PCT No.: PCT/US2024/026758

§ 371 Date: Nov. 4, 2025

Related U.S. Application Data
(60) Provisional application No. 63/466,794, filed on May 16, 2023.

(51) Int. Cl. G16B 15/20; G16B 30/00; G16B 40/20

(52) U.S. Cl. CPC G16B 15/20; G16B 30/00; G16B 40/20

(57) ABSTRACT

Disclosed are various embodiments for antibody clearance prediction using antibody sequence properties and machine learning techniques. In particular, an antibody clearance can be predicted using physicochemical features (e.g., structure, charge, moment, etc.) derived from biophysical molecular models and protein language model (pLM) embeddings derived from trained deep-learning protein language models (pLMs). A trained clearance prediction model predicts nonspecific clearance of a given antibody sequence according to the physicochemical features and the embeddings.

### CROSS REFERENCE TO RELATED CASES

This application claims the benefit of and priority to U.S. Patent Application No. 63/466,764 filed on 16 May 2023, entitled “CLEARANCE PREDICTION ACCORDING TO ANTIBODY PROPERTY ANALYSIS,” the contents of which are incorporated by reference in their entirety herein.

### BACKGROUND

Monoclonal antibodies (mAbs) are a valuable and widely used format for therapeutic drugs. While mAbs typically require intravenous or subcutaneous administration, their long, systemic persistence enables less frequent dosing than small molecules. Antibodies with atypically fast clearance rates require more frequent dosing, limiting clinical developability and utility. Animal models and assay based approaches are often used to anticipate such liabilities prior to clinical development, with clearance in cynomolgus monkeys (cyno) being one of the best predictors of nonspecific clearance in humans.

### BRIEF DESCRIPTION OF THE DRAWINGS

Many aspects of the present disclosure can be better understood with reference to the following drawings. The components in the drawings are not necessarily to scale, with emphasis instead being placed upon clearly illustrating the principles of the disclosure. Moreover, in the drawings, like reference numerals designate corresponding parts throughout the several views.

FIG. 1 is a drawing depicting one of several embodiments of the present disclosure.

FIG. 2 is a drawing of a network environment according to various embodiments of the present disclosure.

FIG. 3A is an example pictorial diagram of a deep learning neural network corresponding to the feature representation model of FIGS. 1 and 2 according to various embodiments of the present disclosure.

FIGS. 3B and 3C are example schematic representations of physicochemical features that can be included in an output of the biophysical model of FIGS. 1 and 2 according to various embodiments of the present disclosure.

FIG. 4 is a flowchart illustrating one example of functionality implemented as portions of an application executed in a computing environment in the network environment of FIG. 2 according to various embodiments of the present disclosure.

FIG. 5 is a flowchart illustrating one example of functionality implemented as portions of an application executed in a computing environment in the network environment of FIG. 2 according to various embodiments of the present disclosure.

FIG. 6 is a flowchart illustrating one example of functionality implemented as portions of an application executed in a computing environment in the network environment of FIG. 2 according to various embodiments of the present disclosure.

### DETAILED DESCRIPTION

The present disclosure relates to antibody clearance prediction using antibody sequence properties and machine learning techniques. In particular, the present disclosure relates to predicting an antibody clearance using physicochemical features (e.g., structure, charge, moment, etc.) derived from biophysical molecular models and protein language model (pLM) embeddings derived from trained deep-learning protein language models (pLMs). According to various examples, a clearance prediction model can be trained to predict a clearance for a given antibody using a dataset of antibody sequences and experimentally measured nonspecific clearances for the antibody sequences. The trained clearance prediction model can be used to predict nonspecific clearance of a given antibody sequence according to an analysis of the antibody sequence properties prior to or after the generation of antibodies (e.g., antibodies and antibody clones) associated with the antibody sequence and prior to in vitro or animal testing. In various examples, the clearance prediction model can be trained using clearance rates and/or thresholds associated with cynos, humans, other species (e.g., murine species, etc.), assays, and/or a combination thereof.

Monoclonal antibodies (mAbs) are typically administered intravenously or subcutaneously to avoid degradation. The long systemic persistence (i.e., long half-life, low clearance) of mAbs, due in part to recycling via the neonatal Fc receptor (FcRn) enables less frequent administration. Antibodies that exhibit unfavorable biophysical properties can exhibit in vivo aggregation, atypical distribution, nonspecific/off-target binding, or other behaviors that accelerate clearance and limit developability and clinical utility.

mAbs for a given target, with similar isotype and backbone, can show significant differences in clearance both in humans and in cynomolgus monkeys (cynos), which are the preferred species for preclinical pharmacokinetics (PK) studies. In order to identify lead candidate antibodies in preclinical research and development, in vitro assays as well as in silico approaches have been previously developed that show association with non-specific clearance measurements in cynos. For example, an assay based on enzyme-linked immunoassay (ELISA) detection of non-specific binding to baculovirus particles identifies mAbs with higher risk of fast clearance. While assays such as this are currently incorporated into standard practices for therapeutic antibody screening and lead generation, they require effort and resources and can only be performed after generation of the molecules. In another example, an in-silico association analysis of a set of mAbs showed that mAbs with faster clearance in cynos tend to have either higher hydrophobicity in complementarity determining regions (CDRs) of their variable domain (Fv), or extreme Fv charge. However, association studies only reveal linear relationship between properties and clearance value, while the promise of machine learning methods is to understand and capture the effect of all features (linear as well as nonlinear) simultaneously. Therefore, an in-silico approach that leverages sequence and structure-based machine learning techniques for nonspecific clearance prediction of mAbs prior to the generation and testing of mAbs can be beneficial.

According to various examples, the present disclosure relies on physicochemical features extracted from molecular structural modeling and/or on feature embeddings derived from protein language models to predict nonspecific clearance for a given antibody sequence. In some embodiments, a clearance prediction model that predicts a clearance for a given antibody sequence can be trained using the physicochemical features and/or the feature embeddings observed from a dataset of antibody sequences and the experimentally measured nonspecific clearances for the antibody sequences. In various examples, the clearance prediction model is trained to maximize prediction accuracy and precision in order to avoid incorrectly classifying potentially promising molecules as fast-clearing (e.g., greater than 8 mL/kg/day in cynos). In various examples, the clearance prediction model can be trained using clearance rates and/or thresholds associated with cynos, humans, other species (e.g., murine species, etc.), pre-clinical assays, and/or a combination thereof. In the example of other species and assays, the clearance rates can be measured and/or predicted, for example, by preclinical models (e.g., SCID or SCID-FcRNtg mouse models, etc.), in vitro PK screening assays (e.g., BV-Elisa, transcytosis/recycling/binding invitro assays, etc.) in combination with in vivo data, and/or other type of measurement or prediction methods of preclinical species and/or assays. It should be noted that the thresholds can differ based on the type of species and/or assay used to train the data. For example, a threshold associated with cynos will differ from the thresholds associated with humans and/or other species and assays.

In various examples, the clearance prediction model can be tuned to minimize false positives (e.g., predicted fast clearances which are actually slow clearances) to avoid eliminating potential sequences that may actually be useful, and/or false negatives (e.g., predicted slow clearances which are actually fast clearances) to reduce risk of advancing fast-clearing molecules. For example, performance metrics (e.g., accuracy, precision, F1, etc.) can be weighted such that false positives or false negatives can be penalized or tolerated based at least in part on the desired performance.

As a given antibody sequence is created, the physicochemical features and/or feature embeddings can be determined according to the antibody sequence and can be used as inputs to the trained clearance prediction model to predict whether the antibody sequence corresponds to a slow clearing or fast clearing antibody. Since the prediction can occur prior to the generation of the corresponding antibody, the time and costs associated with the generation and testing of antibody can be reduced by eliminating the antibody sequences predicted to have fast clearances.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principals disclosed by the following illustrative examples.

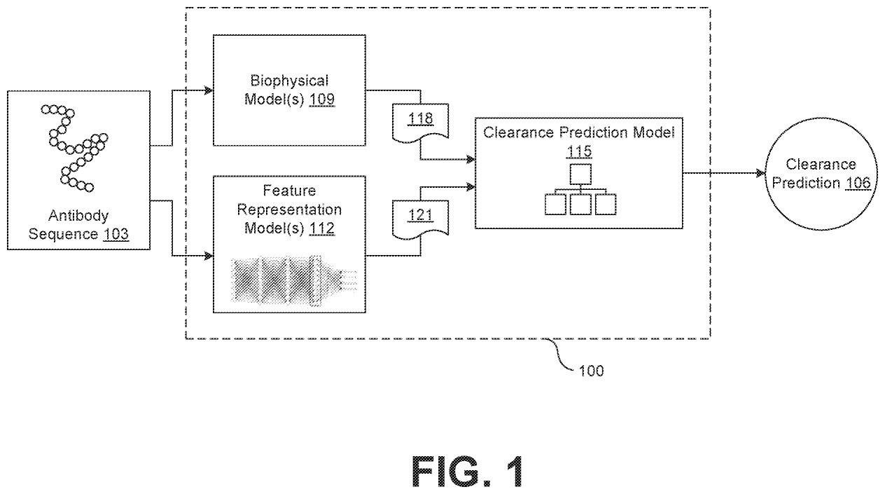

Turning now to FIG. 1, shown is an example schematic drawing illustrating a clearance prediction system 100 that analyzes an antibody sequence 103 and predicts a non-specific clearance for the antibody sequence 103 according to various embodiments. In various examples, the antibody sequence 103 can correspond to a sequence for an antibody (e.g., mAbs) or other type of protein having an Fc region and binds to FcRN that is being considered for generation and subsequent testing. Prior to investing the time and costs to generate and test the corresponding antibody, the antibody sequence 103 can be applied as an input to the clearance prediction system 100, which in turn outputs a clearance prediction 106 associated with a clearance rate for the antibody.

A clearance rate corresponds to a predicted rate of system removal per unit time. In various examples, the clearance prediction 106 can comprise a value that indicates whether an antibody for the given antibody sequence 103 is predicted to have a fast clearance or a slow clearance. For example, the clearance prediction 106 can comprise a discrete value (e.g., 0 or 1) that can represent whether the clearance is a slow clearance (e.g., 0) or a fast clearance (e.g., 1) based at least in part on a comparison to a threshold value (e.g., 8 mL/kg/day in cynos). In other examples, the clearance prediction 106 can correspond to a value corresponding to a probability or level of confidence of a fast clearance or a slow clearance. In this example, the probability or level of confidence value can be compared to a threshold value for classification. In other examples, the clearance prediction 106 can comprise a value that can be representative of a clearance rate or can comprise an actual clearance rate which can then be compared to a threshold value to determine whether the clearance prediction 106 is a fast clearance or a slow clearance.

A fast clearance corresponds to a clearance rate (e.g., rate of system removal per unit time) that is greater than a given threshold value that represents the maximum rate of system removal per unit time deemed acceptable. Similarly, a slow clearance corresponds to a clearance rate that meets or is less than the given threshold value that represents the maximum rate of system removal per unit time deemed acceptable. In some examples, a fast clearance can correspond to greater than 8 mL/kg/day in cynos and a slow clearance can correspond to less than or equal to 8 mL/kg/day in cynos. However, it should be noted that the values for fast and slow clearance are example values and can be modified or adjusted as can be appreciated.

As can be appreciated, an antibody having a fast clearance may be considered undesirable because the antibody would have to be administered to a user with a greater frequency due to the fast rate of removal through the body per unit time. Likewise, an antibody that stays in the system for a longer period of time may be considered desirable due to the reduced frequency for administering to a user. By eliminating fast clearing antibodies prior to generation and testing, time and costs associated with the generation and testing of antibodies can be substantially reduced.

The clearance prediction system 100 can comprise a biophysical model(s) 109, a feature representation model(s) 112, and a clearance prediction model 115 according to various embodiments. The biophysical model 109 can comprise one or more sequence-based algorithms that estimate molecular structural features associated with an antibody structure through biophysical modeling. In various examples, a biophysical model 109 can include one or more mathematical functions that are configured to determine the physicochemical features 118 (e.g., structure, charge, moment, etc.) of an antibody based at least in part on an analysis of the antibody sequence 103. In various examples, the biophysical models 109 can be generated using a molecular modeling system such as, for example, the Molecular Operating Environment® (MOE®). For example, a molecular modeling system can be used to build homology models that can be used to determine physicochemical features 118 for a given antibody sequence 103.

The physicochemical features 118 that are derived from the biophysical model(s) 109 can comprise features associated with structure, charge, moment, and/or other physicochemical features of the antibody. For example, the physicochemical features 118 can comprise volume, molecular weight, surface area, mobility, positive charge patches, negative charge patches, ionized charged patches, radius of solvent accessible area, net charge, moments (e.g., protein dipole, hydrophobicity, monopole, dipole expansion, advanced expansion, etc.), charges at one or more positions, and/or other types of physicochemical properties 118 that can be determined using biophysical modeling.

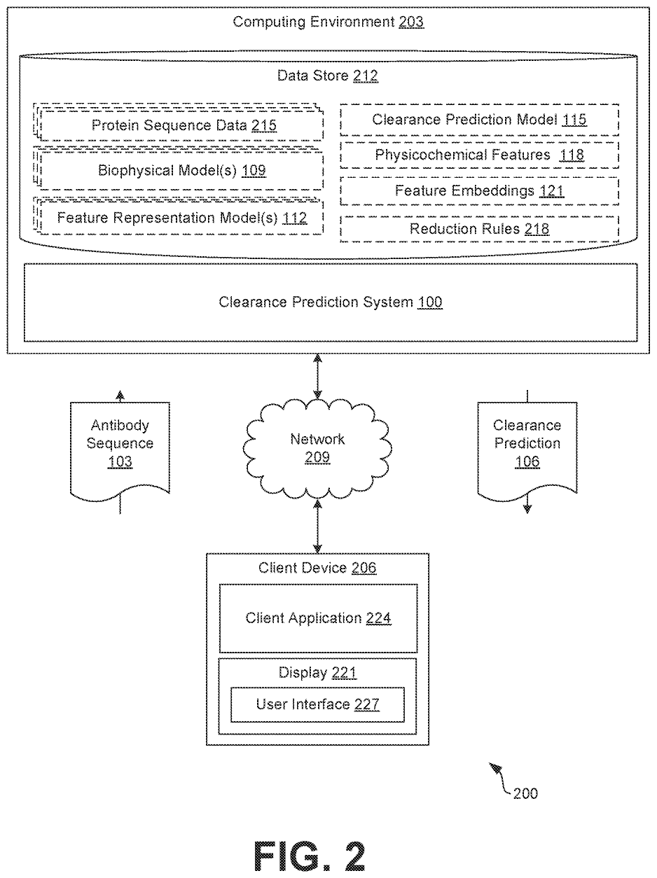

The feature representation model 112 comprises a deep learning neural network model that has been trained to produce feature embeddings 121 associated with a given antibody sequence 103. In particular, the feature representation model 112 leverages models trained on millions of antibody sequences to extract antibody representations from task-agnostic protein language models (pLMs) that can be transferred to antibodies (e.g., mAbs). In various examples, the feature representation model 112 can be trained using an antibody sequence 103 and protein sequence data 215 (FIG. 2) corresponding to a protein family database (e.g., Pfam). During this process, the structural information contained within the antibody sequence 103 is converted into a multi-scale organization vector (e.g., embedding 121) that semantically incorporates the same information. Accordingly, an embedding 121 represents a vector that contains different levels of information from residue-level features to features related to the homology of an antibody. In various examples, the feature representation model 112 can comprise a generative model or deep neural network model (e.g., deep manifold sampler (DMS)) that comprises a transformer-like model pre-trained on sequences from a protein family database (e.g., Pfam) (e.g., protein sequence data 215) and further fine-tuned on a dataset of sequences of antibodies (e.g., sequences from Observed Antibody Space (OAS)) (e.g., protein sequence data 215). The antibody sequence 103 can be applied as an input to the feature representation model 112, which processes the antibody sequence 103 through a plurality of hidden layers and ultimately yields a multi-feature embedding 121 representing encoded features for the entire antibody molecule associated with the antibody sequence 103.

The clearance prediction model 115 can include, for example, a decision tree classifier, a gradient boost classifier, a Gaussian naïve Bayes classifier, a reinforcement learning algorithm, a logistic regression classifier, a random forest classifier, a multi-layer perceptron classifier, a recurrent neural network, a neural network, a label-specific attention network, an ensemble model, and/or any other type of trained model as can be appreciated. Although the clearance prediction model 115 can comprise a neural network, it is noted that the computational overhead associated with the neural network can be minimized by implementing a classifier model that is not a neural network.

In various examples, the clearance prediction model 115 is able to capture complex relationships between input variables and can perform accurately with small datasets (e.g., about 100 datapoints). For example, the clearance prediction model 115 can comprise a gradient boost classifier which is known to be flexible and capture complex relationships between variables. In addition, the gradient boost classifier can perform well with small datasets while other models may be prone to overfitting when subject to smaller datasets which can negatively impact model performance when exposed to new data.

In various examples, the clearance prediction model 115 is trained to predict a clearance for a given antibody based at least in part on physicochemical features 118 and/or feature embeddings 121 that are derived from an antibody sequence 103. The output of the clearance prediction model 115 includes the clearance prediction 106 associated with a clearance rate for the antibody associated with the given antibody sequence 103. The clearance rate corresponds to a predicted rate of system removal per unit time. In various examples, the clearance prediction 106 can comprise one or more values that can represent whether an antibody for the given antibody sequence 103 is predicted to have a fast clearance or a slow clearance. For example, the clearance prediction 106 can comprise a discrete value (e.g., 0 or 1) that can represent whether the clearance is a slow clearance (e.g., 0) or a fast clearance (e.g., 1) based at least in part on a comparison to a threshold value (e.g., 8 mL/kg/day in cynos). In other examples, the clearance prediction 106 can correspond to a probability or level of confidence of a fast clearance or a slow clearance. In this example, the probability or level of confidence can be compared to a threshold value for classification. In other examples, the clearance prediction 106 can comprise a value that can be representative of a clearance rate or can comprise an actual clearance rate which can then be compared to a threshold value to determine whether the clearance prediction 106 is a fast clearance or a slow clearance.

A fast clearance corresponds to a clearance rate (e.g., rate of system removal per unit time) that is greater than a given threshold value that represents the maximum desired rate of system removal per unit time. Similarly, a slow clearance corresponds to a clearance rate that meets or is less than the given threshold value that represents the maximum desired rate of system removal per unit time. In some examples, a fast clearance can correspond to greater than 8 mL/kg/day in cynos and a slow clearance can correspond to less than or equal to 8 mL/kg/day in cynos. However, it should be noted that the values for fast and slow clearance are example values and can be modified or adjusted as can be appreciated.

In various examples, the clearance prediction model can be trained using clearance rates and/or thresholds associated with cynos, humans, other species (e.g., murine species, etc.), assays, and/or a combination thereof. In the example of other species and assays, the clearance rates can be measured and/or predicted, for example, by data from preclinical models (e.g., SCID or SCID-FcRNtg mouse models, etc.), in vitro PK screening assays (e.g., BV-Elisa, transcytosis/recycling/binding invitro assays, etc.) in combination with in vivo data, and/or other type of measurement or prediction methods of preclinical species and/or assays. It should be noted that the thresholds can differ based on the type of species and/or assay used to train the data. For example, a clearance threshold associated with cynos will differ from the clearance thresholds associated with humans and/or other species and assays.

Further, in various examples, the clearance prediction model 115 can be tuned to minimize false positives (e.g., predicted fast clearances which are actually slow clearances) to reduce the elimination of potential sequences 103 that may actually be useful prior to further generation and testing of the antibodies associated with the antibody sequences 103. For example, performance metrics (e.g., accuracy, precision, F1, etc.) can be weighted such that false positives or false negatives can be penalized or tolerated based at least in part on the desired performance.

It should be noted that although FIG. 1 illustrates the clearance prediction system 100 as including both a biophysical model 109 and a feature representation model 112, in various examples, the clearance prediction model 115 can comprise biophysical model(s) 109 or feature representation model(s) 112. For example, in some embodiments, the clearance prediction model 115 can be trained using both the physicochemical features 118 that are derived from the biophysical model(s) 109 and the feature embeddings 121 that are derived from the feature representation model(s) 112. In this example, the clearance prediction system 100 includes both a biophysical model 109 and a feature representation model 112, as illustrated in FIG. 1. In other embodiments, the clearance prediction model 115 can be trained using the physicochemical features 118 that are output from the biophysical models 109 but not the feature embeddings 121. In this example, the clearance prediction system 100 includes a biophysical model 109 but not a feature representation model 112. In other embodiments, the clearance prediction model 115 can be trained using the feature embeddings 121 that are derived from a feature representation model 112 but not the physicochemical features 118 derived from a biophysical model 109. In this example, the clearance prediction system 100 includes a feature representation model 112 but not a biophysical model 109.

Turning now to FIG. 2, shown is a network environment 200 according to various embodiments. The network environment 200 can include a computing environment 203, and a client device 206, which can be in data communication with each other via a network 209.

The network 209 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 209 can also include a combination of two or more networks 209. Examples of networks 209 can include the Internet, intranets, extranets, virtual private networks (VPNs), and similar networks.

The computing environment 203 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 203 can employ a plurality of computing devices that can be arranged in one or more server banks or computer banks or other arrangements. Such computing devices can be located in a single installation or can be distributed among many different geographical locations. For example, the computing environment 203 can include a plurality of computing devices that together can include a hosted computing resource, a grid computing resource, an edge computing resource, or any other distributed computing arrangement. In some cases, the computing environment 203 can correspond to an elastic computing resource where the allotted capacity of processing, network, storage, or other computing-related resources can vary over time.

Various applications or other functionality can be executed in the computing environment 203. The components executed on the computing environment 203 include the clearance prediction system 100, and other applications, services, processes, systems, engines, or functionality not discussed in detail herein.

The clearance prediction system 100 can be executed to analyze antibody sequences 103 to predict a clearance prediction 106 for an antibody according to various embodiments of the present disclosure. In particular, the clearance prediction system 100 can be executed to obtain an antibody sequence 103 and apply the antibody sequence 103 as an input to a biophysical model 109 and/or a feature representation model 112. As described, the biophysical model 109 can include one or more mathematical functions that are configured to determine the physicochemical features 118 (e.g., structure, charge, moment, etc.) of an antibody based at least in part on an analysis of the antibody sequence 103. The output of the biophysical model 109 can comprise physicochemical features 118 associated with structure, charge, moment, and or other physicochemical features of an antibody according to the analysis of the antibody sequence 103. The clearance prediction system 100 can obtain the physicochemical features 118 from the output of the biophysical model 109. In some examples, the clearance prediction system 100 can reduce the physicochemical features 118 by selecting a subset based at least in part on a clearance prediction effect analysis (e.g., a feature importance perturbation analysis).

The feature representation model 112 comprises a deep learning neural network model that produces feature embeddings 121 associated with the inputted antibody sequence 103. The clearance prediction system 100 can obtain the feature embeddings 121 that are derived from the feature representation model 112. In some examples, the clearance prediction system 100 can further reduce the feature embeddings 121 using a reduction approach such as, for example, PCA. The reduction number can be based on for example a predefined value.

The clearance prediction system 100 can get the features (e.g., feature embeddings 121, physicochemical features 118) derived from the feature representation model 112 and/or the biophysical model 109 and apply the features as inputs to the clearance prediction model 115. The clearance prediction system 100 can obtain the clearance prediction 106 that is output from the clearance prediction model 115. In various examples, the clearance prediction system 100 can generate a notification including the clearance prediction 106 associated with the antibody sequence 103 and provide to the client device 206. Accordingly, a user associated with the client device 206 can obtain the clearance prediction 106 and determine whether to eliminate the antibody sequence 103 from further evaluation (e.g., generation, testing). For example, if the clearance prediction 106 indicates a fast clearance, the antibody associated with that sequence 103 may not be suitable for further development and evaluation.

In various examples, the clearance prediction system 100 can be executed to train the clearance prediction model 115. The clearance prediction model 115 can include, for example, a decision tree classifier, a gradient boost classifier, a Gaussian naïve Bayes classifier, a reinforcement learning algorithm, a logistic regression classifier, a random forest classifier, a multi-layer perceptron classifier, a recurrent neural network, a neural network, a label-specific attention network, an ensemble model, and/or any other type of trained model as can be appreciated. In various examples, the clearance prediction model 115 is trained to predict a clearance for a given antibody based at least in part on physicochemical features 118 and/or feature embeddings 121 that are derived from a given antibody sequence 103. The output of the clearance prediction model 115 includes the clearance prediction 106, which indicates a fast clearance or a slow clearance for the given antibody sequence 103.

In various examples, the clearance prediction system 100 can be executed to obtain a dataset of antibody sequences 103 and experimentally measured nonspecific clearances for the antibody sequences 103. In various examples, the clearance prediction system 100 can split the data into a testing dataset and a training dataset. Accordingly, the clearance prediction system 100 can train the clearance prediction model 115 using the training data set and evaluate the accuracy of the clearance prediction system 100 using the testing dataset and the experimentally measured nonspecific clearances for the antibody sequences 103 included in the testing dataset. In various examples, the clearance prediction system 100 can apply the antibody sequences 103 from the training dataset and the testing dataset to the feature representation model(s) 112 and/or the biophysical model(s) 109 to obtain the derived feature embeddings 121 and/or the physicochemical features 118, which are used to train the clearance prediction model 115.

It should be noted that in some examples, the clearance prediction system 100 does not train the clearance prediction model 115. Accordingly, although not shown in FIG. 2, the computing environment 203 can comprise a training system that is configured to train the clearance prediction model 115 independently of the clearance prediction system 100.

Also, various data is stored in a data store 212 that is accessible to the computing environment 203. The data store 212 can be representative of a plurality of data stores 212, which can include relational databases or non-relational databases such as object-oriented databases, hierarchical databases, hash tables or similar key-value data stores, as well as other data storage applications or data structures. Moreover, combinations of these databases, data storage applications, and/or data structures may be used together to provide a single, logical, data store. The data stored in the data store 212 is associated with the operation of the various applications or functional entities described below. This data can include protein sequence data 215, one or more biophysical models 109, one or more feature representation models 112, a clearance prediction model 115, physicochemical features 118, feature embeddings 121, reduction rules 218, and potentially other data.

The protein sequence data 215 can include antibody sequences 103 that represent sequences for antibodies (e.g., mAbs). In some examples, the antibody sequences 103 correspond to sequences for antibodies that are being considered for generation and subsequent testing. In other examples, the antibody sequences 103 are included in a dataset of antibody sequences 103 for antibodies that have been previously generated and experimentally evaluated. In the example of experimentally tested antibody sequences 103, the protein sequence data 215 can further include experimentally measured nonspecific clearances for the antibody sequences 103 included in the dataset. The protein sequence data 215 can further include sequence data from a protein family database (e.g., Pfam). The protein sequence data 215 corresponding to the protein family database can be used to train the feature representation models 112 to output the desired embeddings 121.

The biophysical model 109 can comprise one or more sequence-based algorithms that predict molecular structural features associated with an antibody through biophysical modeling. In various examples, a biophysical model 109 can include one or more mathematical functions that are configured to determine the physicochemical features 118 (e.g., structure, charge, moment, etc.) of an antibody based at least in part on an analysis of the antibody sequence 103. In various examples, the biophysical models 109 can be generated using a molecular modeling system such as, for example, the Molecular Operating Environment® (MOE®). For example, a molecular modeling system can be used to build homology models that can be used to determine physicochemical features 118 for a given antibody sequence 103. In various examples, the physicochemical features 118 can include antibody structure features in the Fab domains as well as electrostatic multipole moments and charge features of full-length antibodies. In various examples, a biophysical model 109 can be configured to output the physicochemical features 118 at varying pH levels. For example, the physicochemical features 118 can be calculated at pH 5.5, pH 7.4, and/or other PH level. In some examples, the set of physicochemical features 118 selected by the clearance prediction system 100 can be based at least in part on the pH level value associated with the physicochemical features 118.

The feature representation model 112 comprises a deep learning neural network model that has been trained to produce feature embeddings 121 associated with a given antibody sequence 103. In particular, the feature representation model 112 leverages models trained on millions of antibody sequences to extract antibody representations from task-agnostic protein language models (pLMs) that can be transferred to antibodies (e.g., mAbs). During this process, the structural information contained within the antibody sequence 103 is converted into a multi-scale organization vector (e.g., embedding 121) that semantically incorporates the same information. Accordingly, an embedding 121 represents a vector that contains different levels of information from residue-level features to features related to the homology of an antibody. In various examples, the feature representation model 112 can comprise a generative model or deep neural network model (e.g., deep manifold sampler (DMS)) that includes a transformer-like model pre-trained on sequences from a protein family database (e.g., Pfam) and further fine-tuned on a dataset of sequences of antibodies (e.g., sequences from Observed Antibody Space (OAS)). The antibody sequence 103 can be applied as an input to the feature representation model 112, which processes the antibody sequence 103 through a plurality of hidden layers and ultimately yields a multi-feature embedding 121 representing encoded features for the entire antibody molecule associated with the antibody sequence 103.

The clearance prediction model 115 comprises a classifier trained to predict a clearance for a given antibody based at least in part on physicochemical features 118 and/or feature embeddings 121 that are derived from an antibody sequence 103. The output of the clearance prediction model 115 includes the clearance prediction 106, which indicates a fast clearance or a slow clearance for the given antibody sequence 103. The clearance prediction model 115 can include, for example, a decision tree classifier, a gradient boost classifier, a Gaussian naïve Bayes classifier, a reinforcement learning algorithm, a logistic regression classifier, a random forest classifier, a multi-layer perceptron classifier, a recurrent neural network, a neural network, a label-specific attention network, an ensemble model, and/or any other type of trained model as can be appreciated. Although the clearance prediction model 115 can comprise a neural network, it is noted that the computational overhead associated with the neural network can be minimized by implementing classifier model that is not a neural network.

In various examples, the clearance prediction model 115 is able to capture complex relationships between input variables and can perform accurately with small datasets (e.g., about 100 datapoints). For example, the clearance prediction model 115 can comprise a gradient boost classifier which is known to be flexible and capture complex relationships between variables. In addition, the gradient boost classifier can perform well with small datasets while other models may be prone to overfitting when subject to smaller datasets which can negatively impact model performance when exposed to new data.

The physicochemical features 118 include features that are derived from the biophysical model(s) 109 and are associated with structure, charge, moment, and/or other physicochemical features of an antibody associated with a given antibody sequence 103. For example, the physicochemical features 118 can comprise volume, molecular weight, surface area, mobility, positive charge patches, negative charge patches, ionized charged patches, radius of solvent accessible area, net charge, moments (e.g., protein dipole, hydrophobicity, monopole, dipole expansion, advanced expansion, etc.), charges at one or more positions, and/or other types of physicochemical properties 118 that can be determined using biophysical modeling. In various examples, the physicochemical feature 118 can include a reduced set of physicochemical features 118 from the physicochemical features 118 derived from the output of the biophysical models 109. For example, the reduced set of physicochemical features 118 can include one or more physicochemical features 118 that are selected from the physicochemical features 118 derived from the biophysical model(s) 109.

An embedding 121 represents a vector that contains different levels of information from residue-level features to features related to the homology of an antibody. An embedding 121 corresponds to an output of a feature representation model 112 that has been trained to produce feature embeddings 121 associated with a given antibody sequence 103. For example, the feature representation model 112 leverages models trained on millions of antibody sequences to extract antibody representations from task-agnostic protein language models (pLMs) that can be transferred to antibodies (e.g., mAbs). During this process, the structural information contained within the antibody sequence 103 is converted into a multi-scale organization vector (e.g., embedding 121) that semantically incorporates the same information.

The reduction rules 218 include rules, models, and/or configuration data for the various algorithms or approaches employed by clearance prediction system 100 in reducing the physicochemical features 118 that are derived from the biophysical model(s) 109 and/or the feature embeddings 121 that are derived from the feature representation model(s) 112. For example, the reduction rules 218 can include rules indicating the type of the physicochemical features 118 derived from the biophysical model(s) 109 that should be selected as features to the clearance prediction model 115. In some examples, the rules are based at least in part on a pH setting associated with the biophysical model 109. For example, the types of physicochemical features selected from the physicochemical features 118 derived from a biophysical model 109 that is configured to analyze at a pH of 7.4 can differ from the types of physicochemical features selected from the physicochemical features 118 derived from a biophysical model 109 that is configured to analyze at pH 5.5. The clearance prediction system 100 can determine the appropriate types of physicochemical features to select from based at least in part on the reduction rules 218. In addition, the reduction rules 218 can include threshold values that can be used to determine feature cutoff that can be used to reduce the components included in the feature embeddings 121 that are output from the feature representation model 112.

The client device 206 is representative of a plurality of client devices that can be coupled to the network 209. The client device 206 can include a processor-based system such as a computer system. Such a computer system can be embodied in the form of a personal computer (e.g., a desktop computer, a laptop computer, or similar device), a mobile computing device (e.g., personal digital assistants, cellular telephones, smartphones, web pads, tablet computer systems, music players, portable game consoles, electronic book readers, and similar devices), media playback devices (e.g., media streaming devices, BluRay® players, digital video disc (DVD) players, set-top boxes, and similar devices), a videogame console, or other devices with like capability. The client device 206 can include one or more displays 221, such as liquid crystal displays (LCDs), gas plasma-based flat panel displays, organic light emitting diode (OLED) displays, electrophoretic ink (“E-ink”) displays, projectors, or other types of display devices. In some instances, the display 221 can be a component of the client device 206 or can be connected to the client device 206 through a wired or wireless connection.

The client device 206 can be configured to execute various applications such as a client application 224 or other applications. The client application 224 can be executed in a client device 206 to access network content served up by the computing environment 203 or other servers, thereby rendering a user interface 227 on the display 221. To this end, the client application 224 can include a browser, a dedicated application, or other executable, and the user interface 227 can include a network page, an application screen, or other user mechanism for obtaining user input. The client device 206 can be configured to execute applications beyond the client application 224 such as email applications, social networking applications, word processors, spreadsheets, or other applications.

Next, a general description of the operation of the various components of the network environment 200 and the clearance prediction system 100 is provided. To begin, a user interacting with a client application 224 on a client device 206 can send a request to the clearance prediction system 100 to evaluate an antibody sequence 103 and predict a clearance prediction 106 of the antibody sequence 103. Upon receiving the antibody sequence 103, the clearance prediction system 100 can obtain the corresponding biophysical model(s) 109 and/or feature representation model(s) 112 from the data store 212 and apply the antibody sequence 103 as inputs to the biophysical model(s) 109 and the feature representation model(s) 112.

The biophysical model(s) 109 determines the physicochemical features 118 (e.g., structure, charge, moment, etc.) of an antibody by applying the antibody sequence 103 to one or more sequence-based algorithms that predict molecular structural features associated with an antibody structure through biophysical modeling. The physicochemical features 118 that are derived from the biophysical model(s) 109 can comprise features associated with structure, charge, moment, and/or other physicochemical features of the antibody. For example, the physicochemical features 118 can comprise volume, molecular weight, surface area, mobility, positive charge patches, negative charge patches, ionized charged patches, radius of solvent accessible area, net charge, moments (e.g., protein dipole, hydrophobicity, monopole, dipole expansion, advanced expansion, etc.), charges at one or more positions, and/or other types of physicochemical features 118 that can be determined using biophysical modeling.

In some examples, the clearance prediction system 100 can reduce the physicochemical features 118 that are output from the biophysical model(s) 109 prior to providing the physicochemical features 118 as inputs to the clearance prediction model 115. For example, the clearance prediction system 100 can select a portion of the physicochemical features 118 to apply as inputs to the clearance prediction model 115. In some examples, the portion of the physicochemical features 118 selected can be based at least in part on a determined clearance prediction effect associated with the physicochemical features 118. In various examples, the clearance prediction effect can be determined during the testing and training of the clearance prediction model 115.

In various examples, assume that the biophysical models 109 output thirty-three (33) different types of physicochemical features 118 (e.g., structure-based, charge-based, moment-based, etc.). However, during the testing and training of the clearance prediction model 115, the performance results (e.g., accuracy, precision, recall, etc.) of the clearance predicting model 115 may indicate that that only fifteen (15) of the types of physicochemical features 118 had a meaningful effect on the clearance prediction. In various examples, a list of the physicochemical features 118 that that were determined during the testing and training of the clearance prediction model 115 to have a meaningful effect on the clearance prediction 109 can be defined. In some examples, the list of physicochemical features 118 can be included in the reduction rules 218 used by the clearance prediction system 100 to select the portion of the physicochemical features 118 that are to be used as inputs to the trained clearance prediction model 115. As such, in this example, the clearance prediction system 100 may select a portion of physicochemical features 118 including only those fifteen (15) physicochemical features 118 (e.g., defined in the reduction rules 218) to be applied as inputs to the clearance prediction model 115.

In various examples, the clearance prediction system 100 can use the rules and configurations included in the reduction rules 218 to define how the physicochemical features 118 are to be selected. In various examples, the reduction rules 218 can be user defined and/or dynamically generated based at least in part on observance by the clearance prediction system 100. By using a reduced portion of the physicochemical features 118 as inputs to the clearance prediction model 115 for training or actual use, the training of the clearance prediction model 115 can be improved and the computing resources and overall computing overhead associated with the training and use of the clearance prediction model 115 can be reduced.

The feature representation model(s) 112 comprises a deep learning neural network model that has been trained to produce feature embeddings 121 associated with a given antibody sequence 103. Accordingly, the feature representation model 112 will generate the feature embeddings 121 representing encoded features of the given antibody sequence 103 in accordance with the various layers of the trained deep learning neural network and protein language models (pLMs). In various examples, the clearance prediction system 100 can further reduce the feature embeddings 121 that are output from the feature representation model 112. For example, assume that the feature representation model 112 yielded a 256 feature embedding for the antibody sequence 103 with the heavy and light chains of the antibody being embedded together. These features may contain some redundancy. As such, the features of the feature embeddings 121 can be further reduced to minimize the redundancy and/or to provide a more manageable data set to improve overall computing performance. In various examples, principal components analysis (PCA) can be used for feature reduction of the feature embeddings 121. In various examples, the clearance prediction system 100 can use the reduction rules 218 to define how the feature embeddings 121 are to be reduced.

In response to obtaining and, if applicable, reducing the physicochemical features 118 derived from biophysical model(s) 109 and the feature embeddings 121 derived from the feature representation model(s) 112, the clearance prediction system 100 can apply a hybrid of the physicochemical features 118 and the feature embeddings 121 as inputs to the trained clearance prediction model 115. Accordingly, the trained clearance prediction model 115 analyzes the inputted features and generates the clearance prediction 106 as an output. Upon receiving the output, the clearance prediction system 100 can generate a notification including the clearance prediction 106 that can be used to determine whether to continue with the generation and testing process of the antibody associated with the given antibody sequence 103. The clearance prediction system 100 can transmit the notification to client device 206 for rendering on a user interface 227 and review by a user interacting with the client device 206.

In accordance to various embodiments, the time and costs associated with the generation and testing of antibodies can be substantially reduced by eliminating fast clearing antibodies in response to identifying an antibody sequence 103, determining antibody properties associated with the antibody sequence 103 (e.g., physicochemical features 118 and feature embeddings 121), and determining a clearance prediction for the antibody prior to generation and testing of the antibody. In addition, the clearance prediction model 115 can be tuned to minimize false positives (e.g., predicted fast clearances which are actually slow clearances) and/or false negatives (e.g., predicted slow clearances which are actually fast clearances) to avoid eliminating potential sequences 103 that may actually be favorable for use. For example, performance metrics can be weighted such that false positives or false negatives can be penalized or favorited based at least in part on the desired performance.

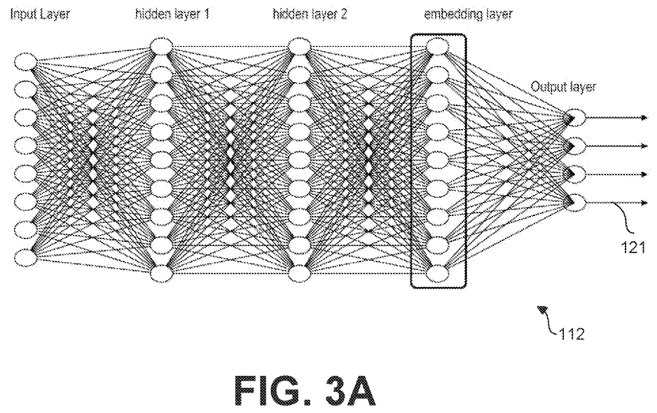

Referring next to FIG. 3A, shown is an example schematic of a deep learning neural network that corresponds to the trained feature representation model 112 of the present disclosure in accordance to various embodiments. In various examples, the feature representation model 112 can comprise a generative model or deep neural network model (e.g., deep manifold sampler (DMS)) that includes a transformer-like model pre-trained on sequences from a protein family database (e.g., Pfam) and further fine-tuned on a dataset of paired sequences of antibodies (e.g., sequences from Observed Antibody Space (OAS)). An antibody sequence 103 can be applied as an input to the feature representation model 112, which processes the antibody sequence 103 through a plurality of hidden layers and ultimately yields a multi-feature embedding 121 representing encoded features for the entire antibody molecule associated with the antibody sequence 103.

As illustrated in FIG. 3A, the feature representation model 112 can comprise an input layer, one or more hidden layers, an embedding layer, and an output layer. As the feature representation model 112 analyzes the input antibody sequence 103, the structural information contained within the antibody sequence 103 is converted into a multi-scale organization vector (e.g., embedding 121) that semantically incorporates the same information. The embedding 121 is included in the output of the feature representation model 112.

Turning now to FIGS. 3B and 3C, shown are example schematic representations of some of the physicochemical features 118 or descriptors that can be included in an output of a biophysical model 109. For example, FIG. 3B illustrates an example of a physicochemical property that corresponds to Fab-level protein descriptors that can be generated from a molecular modeling application that can be used to define the biophysical model 109. In particular, FIG. 3B illustrates example representations of a hydrophobic patch and a negative patch that can be included in the structural representation of the antibody associated with the antibody sequence 103. For example, a hydrophobic patch on an antibody corresponds to clusters of neighboring or otherwise adjacent non-polar atoms that are included on the antibody surface of a given antibody. The negative patch can correspond to a negatively charged patch of neighboring or other adjacent atoms that are included on the antibody surface. FIG. 3C illustrates an example schematic representation of a full-length antibody electrostatic descriptors that can be included in the physicochemical features 118. In particular, FIG. 3C illustrates electrostatic multipole moments up to the octupole order as well as a coarse-grained representation of the charges associated with the antibody.

The example representations shown in FIGS. 3B and 3C of the physicochemical features 118 that can be defined by the biophysical model 109 are merely examples. In particular, the physicochemical features 118 can comprise volume, molecular weight, surface area, mobility, positive charge patches, negative charge patches, ionized charged patches, radius of solvent accessible area, net charge, moments (e.g., protein dipole, hydrophobicity, monopole, dipole expansion, advanced expansion, etc.), charges at one or more positions, and/or other type of physicochemical property 118 that can be determined using biophysical modeling.

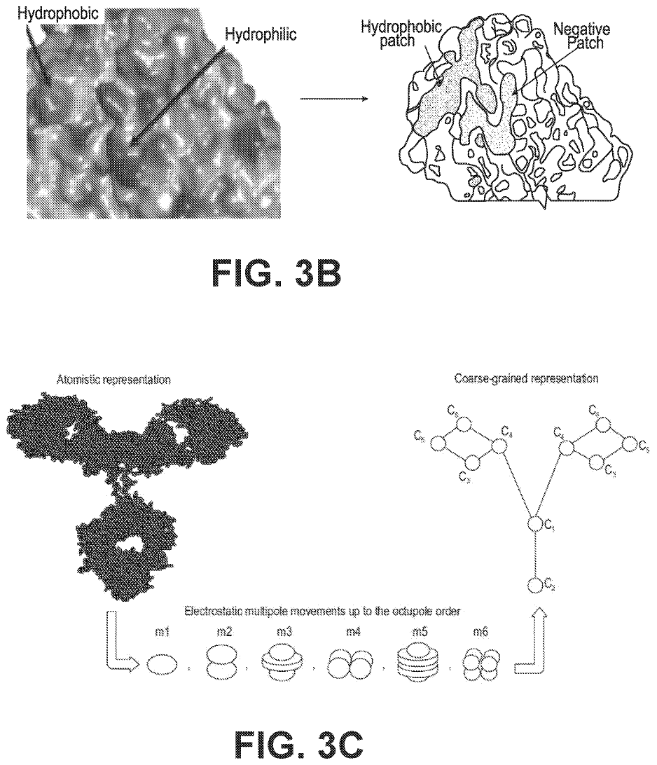

Referring next to FIG. 4, shown is a flowchart 400 that provides one example of the operation of a portion of the clearance prediction system 100. The flowchart 400 of FIG. 4 provides merely an example of the many different types of functional arrangements that can be employed to implement the operation of the depicted portion of the clearance prediction system. As an alternative, the flowchart 400 of FIG. 4 can be viewed as depicting an example of elements of a method implemented within the network environment 200. In particular, FIG. 4 illustrates an example of how the clearance prediction model 115 can be trained by the clearance prediction system 100.

Beginning with block 403, the clearance prediction system 100 obtains protein sequence data 215 including a dataset of antibody sequences 103 and experimentally measured nonspecific clearances for the antibody sequences 103. In various examples, the protein sequence data 215 comprises a dataset of antibody sequences 103 associated with previously generated and evaluated antibody sequences. In various examples, the protein sequence data 215 can be obtained from the data store 212, client device 206, and/or other entity or data store. In various examples, the clearance prediction system 100 can apply the antibody sequences 103 from the training dataset and the testing dataset to the feature representation model(s) 112 and/or the biophysical model(s) 109 to obtain the derived feature embeddings 121 and/or the physicochemical features 118, which are used to train the clearance prediction model 115. Accordingly, the protein sequence data 215 can include the features of the feature embeddings 121 and/or the physicochemical features 118 derived from the feature representation model(s) 112 and/or the biophysical model(s) 109 and subsequently reduced.

At block 406, the clearance prediction system 100 can determine a model pipeline to employ for the testing and generating of a clearance prediction model 115. In particular, to train and determine the best performing model, multiple iterations of testing and training can be performed using various different types of model pipelines. A model pipeline can be based at least in part on a combination of model algorithms, selectors, preprocessing steps and associated hyperparameters that can be used to train a model. In various examples, multiple types of model pipelines can be generated and analyzed to determine the best performing model based at least in part on performance metrics.

At block 409, the clearance prediction system 100 can split the protein sequence data 215 into a testing dataset and a training dataset. For example, the protein sequence data 215 can be split into the testing dataset and the training dataset according to a predefined ratio (e.g., 50/50, 80/20, etc.). Accordingly, the clearance prediction system 100 can train the clearance prediction model 115 using the training data set and evaluate the accuracy of the clearance prediction system 100 using the testing dataset and the experimentally measured nonspecific clearances for the antibody sequences 103 included in the testing dataset.

At block 412, the clearance prediction system 100 can preprocess the protein sequence data 215 included in the training set. In various examples, the clearance prediction system 100 can apply the antibody sequences 103 from the training dataset and the testing data set to the feature representation model(s) 112 and/or the biophysical model(s) 109 to obtain the derived feature embeddings 121 and/or the physicochemical features 118 that are used to train the clearance prediction model 115. Accordingly, the protein sequence data 215 can include the features of the feature embeddings 121 and/or the physicochemical features 118 derived from the feature representation model(s) 112 and/or the biophysical model(s) 109. In various examples, the clearance prediction system 100 can preprocess the protein sequence data 215 by reducing the features, using feature engineering such as, for example, feature reduction, feature transformer, missing value imputation, and feature selection, cross-validation, and/or other types of preprocessing.

At block 415, the clearance prediction system 100 trains the clearance prediction model 115 using the protein sequence data 215 included in the training dataset and the corresponding experimentally measured nonspecific clearances for the antibody sequences 103. The clearance prediction model 115 can include, for example, a decision tree classifier, a gradient boost classifier, a Gaussian naïve Bayes classifier, a reinforcement learning algorithm, a logistic regression classifier, a random forest classifier, a multi-layer perceptron classifier, a recurrent neural network, a neural network, a label-specific attention network, an ensemble model, and/or any other type of trained model as can be appreciated. Although the clearance prediction model 115 can comprise a neural network, it is noted that the computational overhead associated with the neural network can be minimized by implementing a classifier model that is not a neural network. In various examples, training the clearance prediction model 115 can further comprise hyperparameter tuning based at least in part on cross-validation.

At block 418, the clearance prediction system 100 tests the trained clearance prediction model 115 using the protein sequence data 215 included in the testing dataset and the corresponding experimentally measured nonspecific clearances for the antibody sequences 103. In particular, the clearance prediction system 100 can evaluate the accuracy of the trained clearance prediction model 115 based at least in part on a comparison of the experimentally measured nonspecific clearances for the antibody sequences 103 with the clearance predictions 106 that are included as outputs from the trained clearance prediction model 115.

At block 421, the clearance prediction system 100 determines whether to evaluate another dataset split for the given model pipeline. For example, the same model pipeline can be evaluated using a different train/test split of the training dataset and the testing dataset. In various examples, the number of times to re-split the datasets and evaluate the given model pipeline can be based at least in part on a threshold value (e.g., fifty times). If a new dataset split is required, the clearance prediction system 100 will return to block 409. Otherwise, the clearance prediction system 100 will proceed to block 424.

At block 424, the clearance prediction system 100 determines performance metrics associated with each of the dataset split. For example, the performance metrics for each of the dataset splits for a given model pipeline can include an accuracy associated with the training data and testing data for a given model pipeline. In some examples, the performance metrics can comprise an average of performance metrics associated with the various dataset splits and can be recorded in association with the given model pipeline.

At block 427, the clearance prediction system 100 determines if another model pipeline is to be tested and evaluated. If another model pipeline is to be tested and evaluated, the clearance prediction system 100 returns to block 406 where a new model pipeline is generated. Otherwise, the clearance prediction system 100 proceeds to block 430.

At block 430, the clearance prediction system 100 determines the best performing model (e.g., model pipeline) based at least in part on an analysis of the performance metrics. For example, if the average performance metrics of a first model pipeline are greater than the average performance metrics of a second model pipeline, the first model pipeline will be selected and stored for use. Thereafter, this portion of the process proceeds to completion.

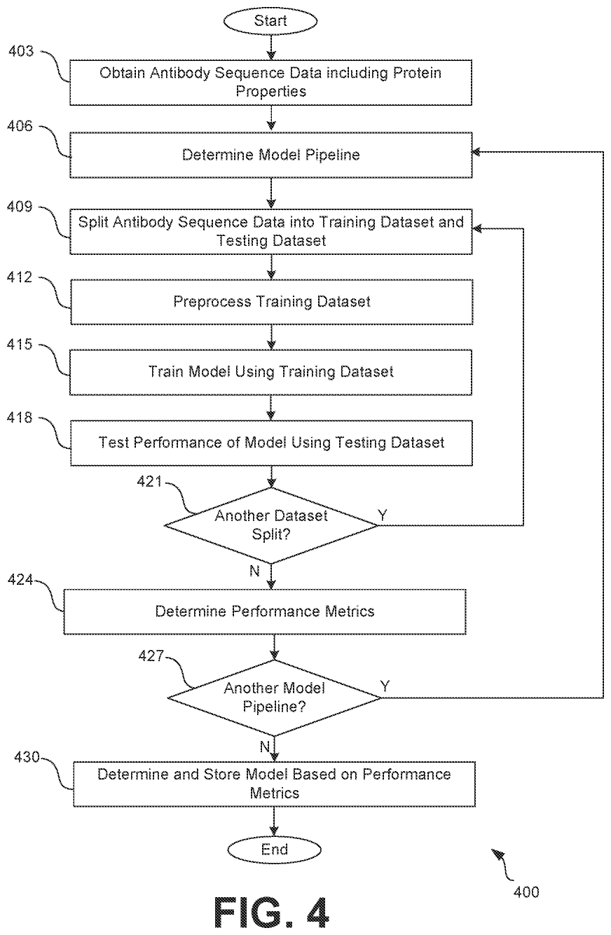

Referring next to FIG. 5, shown is a flowchart 500 that provides one example of the operation of a portion of the clearance prediction system 100. The flowchart 500 of FIG. 5 provides merely an example of the many different types of functional arrangements that can be employed to implement the operation of the depicted portion of the clearance prediction system. As an alternative, the flowchart 500 of FIG. 5 can be viewed as depicting an example of elements of a method implemented within the network environment 200. In particular, FIG. 5 illustrates an example of how the clearance prediction system 100 predicts a clearance for an antibody sequence 103 according to an analysis of the antibody sequence 103 using a trained clearance prediction model 115.

Beginning with block 503, the clearance prediction system 100 can obtain an antibody sequence 103. For example, a user interacting with a client application 224 on a client device 206 can send a request to the clearance prediction system 100 to evaluate an antibody sequence 103 and predict a clearance prediction 106 of the antibody sequence 103. In other examples, the clearance prediction system 100 can obtain an antibody sequence 103 included in the protein sequence data 215 stored in the data store 212.

At block 506, the clearance prediction system 100 determines the physicochemical features 118 associated with the antibody sequence 103 by applying the antibody sequence 103 as an input to a biophysical model 109. For example, the clearance prediction system 100 can obtain a biophysical model 109 from the data store 212 and apply the antibody sequence 103 as an input to the biophysical model 109. The physicochemical features 118 that are derived from the biophysical model(s) 109 can comprise features associated with structure, charge, moment, and or other physicochemical features of the antibody.

At block 509, the clearance prediction system 100 preprocesses the physicochemical features 118 that are output from the biophysical model 109. For example, the clearance prediction system 100 can select a subset of the physicochemical features 118 to reduce the physicochemical features 118 that are output from the biophysical model 109. For example, the clearance prediction system 100 can select a subset of the physicochemical features 118 based at least in part on a clearance prediction effect. In various examples, the clearance prediction system 100 can use the reduction rules 218 to define how the physicochemical features 118 are to be selected.

At block 512, the clearance prediction system 100 determines the feature embeddings 121 associated with the antibody sequence 103 by applying the antibody sequence 103 as an input to the feature representation model 112. The feature representation model 112 comprises a deep learning neural network configured to generate the feature embeddings 121 representing encoded features of the given antibody sequence 103 in accordance with the various layers of the trained deep learning neural network and protein language models (pLMs).

At block 515, the clearance prediction system 100 reduces the feature embeddings 121 that are included in the output of the feature representation model 112. In particular, the features of the feature embeddings 121 can be further reduced to minimize the redundancy and/or to provide a more manageable dataset to improve overall computing performance. In various examples, principal components analysis (PCA) or other type of reduction process can be used for feature reduction of the feature embeddings 121. In various examples, the clearance prediction system 100 can use the reduction rules 218 to define how the feature embeddings 121 are to be reduced.

At block 518, the clearance prediction system 100 applies a hybrid of the physicochemical features 118 and the feature embeddings 121 as inputs to a trained clearance prediction model 115. Accordingly, the trained clearance prediction model 115 analyzes the inputted features and generates the clearance prediction 106 as an output.

At block 521, the clearance prediction system 100 obtains the clearance prediction 106 included in the output of the trained clearance prediction model 115. For example, the clearance prediction 106 indicates whether an antibody for the given antibody sequence 103 is predicted to have a fast clearance or a slow clearance according to a predicted rate of system removal per unit of time.

At block 524, the clearance prediction system 100 generates a notification including the clearance prediction 106. The clearance prediction system 100 can transmit the notification to client device 206 for rendering on a user interface 227 and review by a user interacting with the client device 206. Accordingly, the clearance prediction 106 can be used to determine whether to continue with the generation and testing process of the antibody associated with the given antibody sequence 103. Thereafter, this portion of the process proceeds to completion.

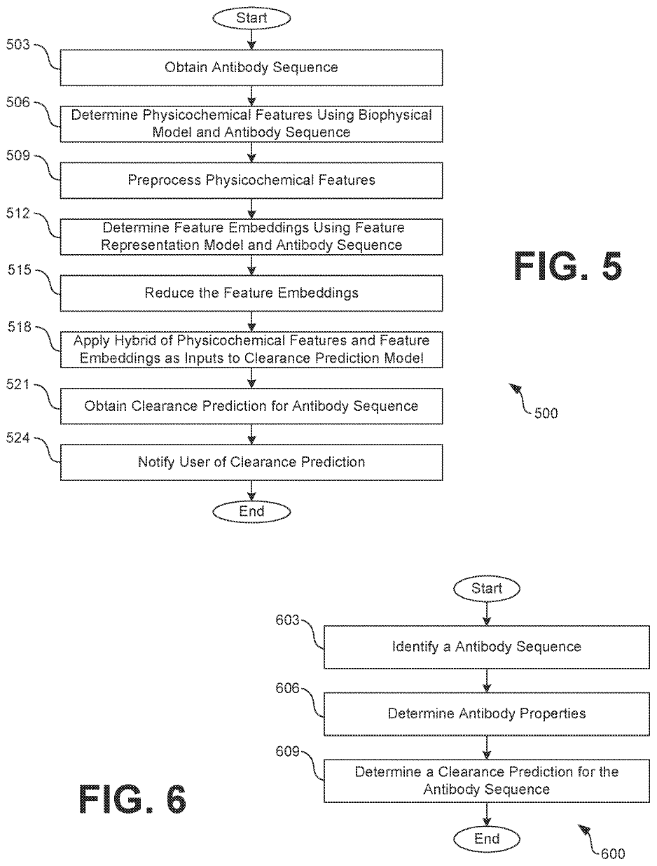

Referring next to FIG. 6, shown is a flowchart 600 that provides one example of the operation of a portion of the clearance prediction system 100. The flowchart of FIG. 6 provides merely an example of the many different types of functional arrangements that can be employed to implement the operation of the depicted portion of the clearance prediction system. As an alternative, the flowchart 600 of FIG. 6 can be viewed as depicting an example of elements of a method implemented within the network environment 200.

In particular, FIG. 6 illustrates an example of how the clearance prediction system 100 predicts a clearance for an antibody sequence 103 according to an analysis of the antibody sequence 103 using a trained clearance prediction model 115. If the clearance is predicted to be a fast clearance, the antibody sequence 103 may be eliminated from further development and testing. By eliminating fast clearing antibodies prior to generation and testing, time and costs associated with the generation and testing of antibodies can be substantially reduced. In addition, the clearance prediction model 115 can be tuned to minimize false positives (e.g., predicted fast clearances which are actually slow clearances) and/or false negatives (e.g., predicted slow clearances which are actually fast clearances) to avoid eliminating potential sequences 103 that may actually be favorable for use. For example, performance metrics can be weighted such that false positives or false negatives can be penalized or favorited based at least in part on the desired performance.

Beginning with block 603, the clearance prediction system 100 identifies an antibody sequence 103. For example, a user interacting with a client application 224 on a client device 206 can send a request to the clearance prediction system 100 to evaluate an antibody sequence 103 and predict a clearance prediction 106 of the antibody sequence 103. The antibody sequence 103 can be included in the request and the clearance prediction system 100 can identify the antibody sequence 103 based at least in part on the request. In other examples, the clearance prediction system 100 can identify the antibody sequence 103 based at least in part on an analysis of the protein sequence data 215 stored in the data store 212.

At block 606, the clearance prediction system 100 determines antibody properties associated with the antibody sequence 103 based at least in part on an analysis of the antibody sequence 103. In various examples, the antibody properties can comprise physicochemical features 118, feature embeddings 121, or a hybrid of physicochemical features 118 and feature embeddings 121. For example, the clearance prediction system 100 can apply the antibody sequence 103 as an input to a biophysical model 109. The output of the biophysical model 109 comprises physicochemical features 118. In some examples, the physiochemical features 118 that are included in the output of the biophysical model 109 can be further reduced by selecting a portion of physicochemical features 118 associated of the antibody that are included in the output of the biophysical model 109. The clearance prediction system 100 determines the feature embeddings 121 associated with the antibody sequence 103 by applying the antibody sequence 103 as an input to the feature representation model 112. In various examples, the features of the feature embeddings 121 can be further reduced to minimize the redundancy and/or to provide a more manageable dataset to improve overall computing performance.

At block 609, the clearance prediction system 100 predicts an antibody clearance (e.g., clearance prediction 106) for the antibody sequence 103. For example, the clearance prediction system 100 can predict the antibody clearance by applying the antibody properties as inputs to a trained clearance prediction model 115. Accordingly, the trained clearance prediction model 115 analyzes the inputted features and generates the clearance prediction 106 as an output. For example, the clearance prediction 106 indicates whether an antibody for the given antibody sequence 103 is predicted to have a fast clearance or a slow clearance according to a predicted rate of system removal per unit of time. Thereafter, this portion of the process proceeds to completion.

A number of software components previously discussed are stored in the memory of the respective computing devices and are executable by the processor of the respective computing devices. In this respect, the term “executable” means a program file that is in a form that can ultimately be run by the processor. Examples of executable programs can be a compiled program that can be translated into machine code in a format that can be loaded into a random access portion of the memory and run by the processor, source code that can be expressed in proper format such as object code that is capable of being loaded into a random access portion of the memory and executed by the processor, or source code that can be interpreted by another executable program to generate instructions in a random access portion of the memory to be executed by the processor. An executable program can be stored in any portion or component of the memory, including random access memory (RAM), read-only memory (ROM), hard drive, solid-state drive, Universal Serial Bus (USB) flash drive, memory card, optical disc such as compact disc (CD) or digital versatile disc (DVD), floppy disk, magnetic tape, or other memory components.

The memory includes both volatile and nonvolatile memory and data storage components. Volatile components are those that do not retain data values upon loss of power. Nonvolatile components are those that retain data upon a loss of power. Thus, the memory can include random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, or other memory components, or a combination of any two or more of these memory components. In addition, the RAM can include static random access memory (SRAM), dynamic random access memory (DRAM), or magnetic random access memory (MRAM) and other such devices. The ROM can include a programmable read-only memory (PROM), an erasable programmable read-only memory (EPROM), an electrically erasable programmable read-only memory (EEPROM), or other like memory device.

Although the applications and systems described herein can be embodied in software or code executed by general purpose hardware as discussed above, as an alternative the same can also be embodied in dedicated hardware or a combination of software/general purpose hardware and dedicated hardware. If embodied in dedicated hardware, each can be implemented as a circuit or state machine that employs any one of or a combination of a number of technologies. These technologies can include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

The flowcharts show the functionality and operation of an implementation of portions of the various embodiments of the present disclosure. If embodied in software, each block can represent a module, segment, or portion of code that includes program instructions to implement the specified logical function(s). The program instructions can be embodied in the form of source code that includes human-readable statements written in a programming language or machine code that includes numerical instructions recognizable by a suitable execution system such as a processor in a computer system. The machine code can be converted from the source code through various processes. For example, the machine code can be generated from the source code with a compiler prior to execution of the corresponding application. As another example, the machine code can be generated from the source code concurrently with execution with an interpreter. Other approaches can also be used. If embodied in hardware, each block can represent a circuit or a number of interconnected circuits to implement the specified logical function or functions.

Although the flowcharts show a specific order of execution, it is understood that the order of execution can differ from that which is depicted. For example, the order of execution of two or more blocks can be scrambled relative to the order shown. Also, two or more blocks shown in succession can be executed concurrently or with partial concurrence. Further, in some embodiments, one or more of the blocks shown in the flowcharts can be skipped or omitted. In addition, any number of counters, state variables, warning semaphores, or messages might be added to the logical flow described herein, for purposes of enhanced utility, accounting, performance measurement, or providing troubleshooting aids, etc. It is understood that all such variations are within the scope of the present disclosure.

Also, any logic or application described herein that includes software or code can be embodied in any non-transitory computer-readable medium for use by or in connection with an instruction execution system such as a processor in a computer system or other system. In this sense, the logic can include statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system. Moreover, a collection of distributed computer-readable media located across a plurality of computing devices (e.g., storage area networks or distributed or clustered filesystems or databases) may also be collectively considered as a single non-transitory computer-readable medium.

The computer-readable medium can include any one of many physical media such as magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium can be a random access memory (RAM) including static random access memory (SRAM) and dynamic random access memory (DRAM), or magnetic random access memory (MRAM). In addition, the computer-readable medium can be a read-only memory (ROM), a programmable read-only memory (PROM), an erasable programmable read-only memory (EPROM), an electrically erasable programmable read-only memory (EEPROM), or other type of memory device.

Further, any logic or application described herein can be implemented and structured in a variety of ways. For example, one or more applications described can be implemented as modules or components of a single application. Further, one or more applications described herein can be executed in shared or separate computing devices or a combination thereof. For example, a plurality of the applications described herein can execute in the same computing device, or in multiple computing devices in the same computing environment 203.

In addition to the foregoing, the various embodiments of the present disclosure include, but are not limited to, the embodiments set forth in the following clauses.

Clause 1. A system, comprising: a computing device comprising a processor and a memory; and machine-readable instructions stored in the memory that, when executed by the processor, cause the computing device to at least: identify an antibody sequence; determine antibody properties associated with the antibody sequence based at least in part on an analysis of the antibody sequence, the antibody properties comprising at least one of: physicochemical features or feature embeddings; and determine a clearance prediction for the antibody sequence by applying the antibody properties as inputs to a trained clearance prediction model.

Clause 2. The system of clause 1, wherein the antibody properties comprise the physicochemical features, and the physicochemical features comprise at least one of a structure-based feature, a moment feature, or a charge feature.

Clause 3. The system of clause 1 or clause 2, wherein, when executed, the machine-readable instructions further cause the computing device to at least determine the physicochemical features by applying the antibody sequence to one or more sequence-based algorithms that predict molecular structural features associated with an antibody structure through biophysical modeling.

Clause 4. The system of clause 3, wherein an output of the one or more sequence-based algorithms comprises the physicochemical features, and, when executed, the machine-readable instructions further cause the computing device to at least: select a portion of the physicochemical features based at least in part on a reduction rule, the portion of the physicochemical features being included in the antibody properties applied as inputs to the trained clearance prediction model.

Clause 5. The system of any one of clauses 1 to 4, wherein the antibody properties comprise the feature embeddings, and when executed, the machine-readable instructions further cause the computing device to at least: apply the antibody sequence as an input to a trained neural network model that outputs the feature embeddings; and reduce the feature embeddings.

Clause 6. The system of clause 5, wherein the feature embeddings are reduced based at least in part on principal component analysis (PCA).

Clause 7. The system of any one of clauses 1 to 6, wherein the antibody properties comprise physicochemical features and feature embeddings.

Clause 8. A method, comprising: identifying an antibody sequence; determining antibody properties associated with the antibody sequence based at least in part on an analysis of the antibody sequence, the antibody properties comprising at least one of: physicochemical features or feature embeddings; and determining a clearance prediction for the antibody sequence by applying the antibody properties as inputs to a trained clearance prediction model.

Clause 9. The method of clause 8, wherein the antibody properties comprise the physicochemical features, and the physicochemical features comprise at least one of a structure-based feature, a moment feature, or a charge feature.

Clause 10. The method of clause 8 or clause 9, further comprising determining the physicochemical features by applying the antibody sequence to one or more sequence-based algorithms that predict molecular structural features associated with an antibody structure through biophysical modeling.

Clause 11. The method of clause 10, wherein an output of the one or more sequence-based algorithms comprises the physicochemical features, and further comprising selecting a portion of the physicochemical features based at least in part on a reduction rule, the portion of the physicochemical features being included in the antibody properties applied as inputs to the trained clearance prediction model.

Clause 12. The method of any one of clauses 8 to 11, wherein the antibody properties comprise the feature embeddings, and further comprising: applying the antibody sequence as an input to a trained neural network model that outputs the feature embeddings; and reducing the feature embeddings to generate the feature embeddings.

Clause 13. The method of clause 12, wherein the feature embeddings are reduced based at least in part on principal component analysis (PCA).

Clause 14. The method of any one of clauses 8 to 13, wherein the antibody properties comprise the physicochemical features and the feature embeddings.

Clause 15. A non-transitory, computer-readable medium, comprising machine-readable instructions that when executed by a processor of a computing device, cause the computing device to at least: identify an antibody sequence; determine antibody properties associated with the antibody sequence based at least in part on an analysis of the antibody sequence, the antibody properties comprising at least one of: physicochemical features or feature embeddings; and determine a clearance prediction for the antibody sequence by applying the antibody properties as inputs to a trained clearance prediction model

Clause 16. The non-transitory, computer-readable medium of clause 15, wherein the antibody properties comprise the physicochemical features, and the physicochemical features comprise at least one of a structure-based feature, a moment feature, or a charge feature.

Clause 17. The non-transitory, computer-readable medium of clause 15 or clause 16, wherein the machine-readable instructions, when executed by the processor, further cause the computing device to at least determine the physicochemical features by applying the antibody sequence to one or more sequence-based algorithms that predict molecular structural features associated with an antibody structure through biophysical modeling.

Clause 18. The non-transitory, computer-readable medium of clause 17, wherein an output of the one or more sequence-based algorithms comprises the physicochemical features, and, when executed, the machine-readable instructions further cause the computing device to at least: select a portion of the physicochemical features based at least in part on a reduction rule, the portion of the physicochemical features being included in the antibody properties applied as inputs to the trained clearance prediction model.

Clause 19. The non-transitory, computer-readable medium of any one of clauses 15 to 18, wherein antibody properties comprise the feature embeddings, and the machine-readable instructions, when executed by the processor, further cause the computing device to at least: apply the antibody sequence as an input to a trained neural network model that outputs the feature embeddings; and reduce the feature embeddings.

Clause 20. The non-transitory, computer-readable medium of any one of clauses 15 to 19, wherein the antibody properties comprise the physicochemical features and the embeddings.

Disjunctive language such as the phrase “at least one of X, Y, or Z,” unless specifically stated otherwise, is otherwise understood with the context as used in general to present that an item, term, etc., can be either X, Y, or Z, or any combination thereof (e.g., X; Y; Z; X or Y; X or Z; Y or Z; X, Y, or Z; etc.). Thus, such disjunctive language is not generally intended to, and should not, imply that certain embodiments require at least one of X, at least one of Y, or at least one of Z to each be present.

It should be emphasized that the above-described embodiments of the present disclosure are merely possible examples of implementations set forth for a clear understanding of the principles of the disclosure. Many variations and modifications can be made to the above-described embodiments without departing substantially from the spirit and principles of the disclosure. All such modifications and variations are intended to be included herein within the scope of this disclosure and protected by the following claims.

1. A system, comprising:
a computing device comprising a processor and a memory; and
machine-readable instructions stored in the memory that, when executed by the processor, cause the computing device to at least:
identify an antibody sequence;
determine antibody properties associated with the antibody sequence based at least in part on an analysis of the antibody sequence, the antibody properties comprising at least one of: physicochemical features or feature embeddings; and
determine a clearance prediction for the antibody sequence by applying the antibody properties as inputs to a trained clearance prediction model.

2. The system of claim 1, wherein the antibody properties comprise the physicochemical features, and the physicochemical features comprise at least one of a structure-based feature, a moment feature, or a charge feature.

3. The system of claim 1, wherein, when executed, the machine-readable instructions further cause the computing device to at least determine the physicochemical features by applying the antibody sequence to one or more sequence-based algorithms that predict molecular structural features associated with an antibody structure through biophysical modeling.

4. The system of claim 3, wherein an output of the one or more sequence-based algorithms comprises the physicochemical features, and, when executed, the machine-readable instructions further cause the computing device to at least: select a portion of the physicochemical features based at least in part on a reduction rule, the portion of the physicochemical features being included in the antibody properties applied as inputs to the trained clearance prediction model.

5. The system of claim 1, wherein the antibody properties comprise the feature embeddings, and when executed, the machine-readable instructions further cause the computing device to at least:
apply the antibody sequence as an input to a trained neural network model that outputs the feature embeddings; and
reduce the feature embeddings.

6. The system of claim 5, wherein the feature embeddings are reduced based at least in part on principal component analysis (PCA).

7. The system of claim 1, wherein the antibody properties comprise physicochemical features and feature embeddings.

8. A method, comprising:
identifying an antibody sequence;
determining antibody properties associated with the antibody sequence based at least in part on an analysis of the antibody sequence, the antibody properties comprising at least one of: physicochemical features or feature embeddings; and
determining a clearance prediction for the antibody sequence by applying the antibody properties as inputs to a trained clearance prediction model.

9. The method of claim 8, wherein the antibody properties comprise the physicochemical features, and the physicochemical features comprise at least one of a structure-based feature, a moment feature, or a charge feature.

10. The method of claim 8, further comprising determining the physicochemical features by applying the antibody sequence to one or more sequence-based algorithms that predict molecular structural features associated with an antibody structure through biophysical modeling.

11. The method of claim 10, wherein an output of the one or more sequence-based algorithms comprises the physicochemical features, and further comprising selecting a portion of the physicochemical features based at least in part on a reduction rule, the portion of the physicochemical features being included in the antibody properties applied as inputs to the trained clearance prediction model.

12. The method of claim 8, wherein the antibody properties comprise the feature embeddings, and further comprising:
applying the antibody sequence as an input to a trained neural network model that outputs the feature embeddings; and
reducing the feature embeddings to generate the feature embeddings.

13. The method of claim 12, wherein the feature embeddings are reduced based at least in part on principal component analysis (PCA).

14. The method of claim 8, wherein the antibody properties comprise the physicochemical features and the feature embeddings.

15. A non-transitory, computer-readable medium, comprising machine-readable instructions that, when executed by a processor of a computing device, cause the computing device to at least:
identify an antibody sequence;
determine antibody properties associated with the antibody sequence based at least in part on an analysis of the antibody sequence, the antibody properties comprising at least one of: physicochemical features or feature embeddings; and
determine a clearance prediction for the antibody sequence by applying the antibody properties as inputs to a trained clearance prediction model.

16. The non-transitory, computer-readable medium of claim 15, wherein the antibody properties comprise the physicochemical features, and the physicochemical features comprise at least one of a structure-based feature, a moment feature, or a charge feature.

17. The non-transitory, computer-readable medium of claim 15 wherein the machine-readable instructions, when executed by the processor, further cause the computing device to at least determine the physicochemical features by applying the antibody sequence to one or more sequence-based algorithms that predict molecular structural features associated with an antibody structure through biophysical modeling.

18. The non-transitory, computer-readable medium of claim 17, wherein an output of the one or more sequence-based algorithms comprises the physicochemical features, and, when executed, the machine-readable instructions further cause the computing device to at least: select a portion of the physicochemical features based at least in part on a reduction rule, the portion of the physicochemical features being included in the antibody properties applied as inputs to the trained clearance prediction model.

19. The non-transitory, computer-readable medium of claim 15, wherein antibody properties comprise the feature embeddings, and the machine-readable instructions, when executed by the processor, further cause the computing device to at least:
apply the antibody sequence as an input to a trained neural network model that outputs the feature embeddings; and
reduce the feature embeddings.

20. The non-transitory, computer-readable medium of claim 15, wherein the antibody properties comprise the physicochemical features and the embeddings.
